(19) United States
(12) Patent Application Publication
(10) Pub. No.: US 2024/0286977 A1
(43) Pub. Date: Aug. 29, 2024

(54) MULTIFUNCTIONAL CATALYST FOR NAPHTHA CRACKING

(71) Applicant: Exelus Inc., Fairfield, NJ (US)

(72) Inventors: Mitrajit Mukherjee, Fairfield, NJ (US); Vamsi M. Vadhri, North Arlington, NJ (US); Narenda Joshi, Jersey City, NJ (US)

(21) Appl. No.: 18/438,462

(22) Filed: Feb. 10, 2024

Related U.S. Application Data
(60) Provisional application No. 63/444,817, filed on Feb. 10, 2023.

(51) Int. Cl. C07C 4/06; B01J 21/04; B01J 29/16; B01J 29/40; B01J 29/48; B01J 29/78; B01J 29/90; B01J 35/40; B01J 35/61; B01J 37/00; B01J 37/02; B01J 37/08; B01J 38/02; B01J 38/12; C10G 11/05; C10G 11/10; C10G 11/18

(52) U.S. Cl. CPC C07C 4/06; B01J 21/04; B01J 29/166; B01J 29/40; B01J 29/48; B01J 29/7815; B01J 29/90; B01J 35/40; B01J 35/615; B01J 35/617; B01J 35/618; B01J 37/0009; B01J 37/0201; B01J 37/0236; B01J 37/08; B01J 38/02; B01J 38/12; C10G 11/05; C10G 11/10; C10G 11/182; B01J 2229/186; B01J 2229/42; C07C 2521/04; C07C 2521/10; C07C 2529/08; C07C 2529/40; C07C 2529/70

(57) ABSTRACT

The addition of small amounts of alkaline earth metal oxides (CaO, MgO) as the isomerization function and transition metal oxides (such as MoO₃, WO₃) as the metathesis function significantly enhances the production of propylene using zeolites. Once the light olefins are formed by cracking on the acid sites of the zeolite, the 1-butene molecules undergo isomerization catalyzed by alkali earth metal oxides and then react with ethylene to produce more propylene.

### RELATED APPLICATIONS

This application claims the priority benefit of U.S. Provisional Patent Application Ser. No. 63/444,817, filed 10 Feb. 2023.

### INTRODUCTION

Light olefins are important raw materials in many petrochemicals because they are building blocks for many useful products. Production of propylene, the second-largest petrochemical after ethylene, has received considerable attention in recent years due to its wide applications as an intermediate in the production of important chemicals such as acrylonitrile, propylene oxide, cumene, acrylic acid and polypropylene. Recently, market analysis showed that the demand for propylene is outpacing that of ethylene and the current supply cannot match the demand.

Propylene is produced largely by steam cracking (SC) of light naphtha (50-60%) and fluid catalytic cracking (FCC) in crude oil refineries (30-40%) and to a lesser extent by olefins metathesis, propane dehydrogenation and methanol to olefins process.

In the past, propylene was produced from steam crackers via heavy liquid cracking and as a result, it was readily available; however, most modern steam crackers use ethane-based feed in place of heavy liquids, leading to less propylene being produced. Hence, SC alone cannot satisfy the demand for propylene. Therefore, there is need of new and improved technologies to produce propylene. While on-purpose propylene production technologies such as propane dehydrogenation and metathesis are viable alternatives, these technologies are costly and hence are less competitive relative to steam crackers and FCC. While the bulk of propylene is produced from SC, SC could be reconfigured to fill the supply-demand gap. However, SC has a number of disadvantages. SC is a non-catalytic, non-selective process and hence it is not flexible. Additionally, it is very energy intensive. In fact, it is the most energy consuming process in the chemical industry accounting for approximately 8% of the total global primary energy use, excluding energy content of final products.

In FCC units, gasoline and/or light olefins are produced via catalytic cracking by contacting a hydrocarbon feedstock with a catalyst usually consisting of crystalline microporous molecular sieves in a circulating fluidized bed. In contrast to SC, FCC technologies have the following advantages:
a) FCC technologies employ catalysts which provide an alternative route to SC needing lower activation energy for C—C bond rupture. This leads to lower operating temperatures (by almost 150-200° C.) than SC processes, leading to large energy savings.
b) The catalysts can be tuned to maximize selectivity to desired products, in this case propylene. Coke formed during the cracking process is constantly removed catalyst regeneration or catalyst decoking.
c) FCC can handle large variation of feed quality by modifying catalyst and operating conditions.

FCC technologies, therefore, are more flexible and can be reconfigured to maximize propylene production and reduce energy consumption by changing the catalyst, feed quality, reactor configuration and process variables.

### BACKGROUND

The FCC process was initially designed to produce gasoline via upgrading low-value feedstocks, such as vacuum gas oil (VGO), and atmospheric residue (AR). The lighter feedstocks such as naphtha need a relatively higher cracking temperature than heavier feedstocks [8]. In FCC, a fluidized-bed (or fluid-bed) of catalyst particles is brought into contact with the gas oil feed along with injected steam at the entrance (called the riser) of the reactor. The hot catalyst particles coming from the regenerator unit evaporate the feed gas oil upon contact in the riser, and the cracking starts as the gas oil vapors and the catalyst particles move upward in the reactor. The temperature of the catalyst particles drops as the evaporation of gas oil and endothermic cracking reactions proceed during the upward movement. Cracking reactions also deposit a significant amount of coke on the catalysts, leading to the deactivation of the catalyst. After removing the adsorbed hydrocarbons by steam stripping, the coked catalyst is sent to the regeneration unit to burn off the coke with air. Heat released from burning the coke deposit increases the temperature of the catalyst particles that are returned to the riser to complete the cycle.

Burning off the rejected carbon (coke) in the regenerator provides the energy necessary for cracking without much loss, thus increasing the thermal efficiency of the process. The cracking products are sent to the fractionator for recovery after they are separated from the catalyst particles in the upper section of the reactor.

The light olefins production research through the catalytic cracking of hydrocarbons such as naphtha started in the late 1960s. In the conventional FCC process, a low amount of olefins are produced (˜1-2 wt. % ethylene and ˜3-6 wt. % propylene). By choosing a proper catalyst and optimizing the operating conditions, this number can be enhanced. Grace Davison developed the Propylene Maximization Catalyst (PMC) catalyst series using a proprietary shape-selective zeolite and matrix technologies, which shows a high propylene yield with low coke formation. Akzo Co. also developed the Advanced Fuels experimental (AFX) series of catalysts as a novel catalytic system containing ex situ phosphorus activated ZSM-5 crystal and claimed a fourfold increase of propylene. The deep catalytic cracking (DCC) process is the extension of FCC, developed by the Research Institute of Petroleum Processing (RIPP) and Sinopec International, which utilizes FCC principles combined with a proprietary catalyst, different operating conditions, and other enhancements for the production of light olefins from VGO. The Indian Oil Corporation's Research and Development Center developed the modified Indmax fluid catalytic cracking (I-FCC) process for the production of light olefins from heavy feedstocks and is able to produce more than 20 wt. % propylene. The high-severity down-flow FCC (HS-FCC), developed by an alliance of Saudi Aramco, King Fahd University of Petroleum and Minerals (KFUPM), and JX Nippon Oil & Energy (JX), can yield up to 25 wt. % propylene through the cracking of heavy hydrocarbons at a temperature of 550° C. to 650° C.

With increasing market demand for propylene and the capability to achieve elevated propylene yields via FCC technology, there is a push to maximize propylene yields. New FCC catalyst technologies involving various methods and configurations are being proposed for enhancing the output of propylene product stream from the FCC unit.

### PRIOR ART

Conventional FCC catalysts consist of an active component (zeolite) which serves as the cracking functions, a matrix which also provides catalytic sites and larger pores both acting as a heat transfer medium and allowing free diffusion of hydrocarbon molecules, a binder (such as bentonite clay) and filler, which provides mechanical strength of the catalyst. Ultra-stabilized zeolite Y (USY) is the zeolite commonly in today's conventional FCC catalyst. The zeolite crystals are dispersed in a matrix of alumina or silica-alumina together with binder and filler material. The matrix can also affect catalyst selectivity, product quality and resistance to poisons. FCC catalysts also have a hierarchical pore architecture containing macro-, meso- and micropores each of which have specific purpose in the overall cracking process.

Traditionally, the FCC unit was developed for the conversion of low value feed into gasoline. However, the unit and the process have undergone several modifications, some of which are specifically aimed to maximize propylene production. Along with the unit and process conditions, even the catalyst has also been redesigned for this purpose.

For this purpose, the catalysts can be acidic, basic, or transition metal oxides. It has been proposed that the catalytic cracking over the basic catalysts proceeds through a free radical mechanism while the catalytic cracking over transition metal oxide catalysts occurs under aerobic conditions and follows a free radical mechanism, where activated oxygen species take hydrogen from hydrocarbons and generate free radicals. The oxidative catalytic cracking can also use the lattice oxygen from the catalyst surface, and this can shift the equilibrium towards products while also reducing the furnace temperature due to the partial supply of heat by combustion reactions. However, these catalysts lose valuable carbon in the form of CO and CO₂. The acidic catalysts showed higher yields of propylene and aromatics and lower ethylene yield at a temperature of 550-650° C. and under the non-aerobic conditions.

For the modified FCC process for maximum propylene production, the desired catalyst properties are:
a) High cracking activity leading to minimal thermal cracking. Thermal cracking leads to undesirable products such as lighter paraffins while catalytic cracking favors light olefins.
b) High selectivity to light olefins, specifically propylene
c) Good hydrothermal stability. Since the units operate at relatively high temperatures (>600° C.) and regeneration produces steam, these catalysts should be resistant to dealumination and deactivation.
d) Large pore sizes for high diffusion of large molecules.
e) High mechanical strength and good attrition resistance so that particle morphology is maintained under the severe impact and stresses that exist in the FCC unit.
f) Low coke production so the catalyst can remain active for a longer period.

Conventionally, zeolite Y was used as a cracking catalyst to convert large vacuum gas oil (VGO) molecules to gasoline range molecules and some amount of light olefins. Later, ZSM-5 was added to the mix to enhance olefin yields. ZSM-5 enhances olefin yields in a twofold mode: one, it consumes carbenium ions generated during primary cracking which initiate the hydrogen transfer mechanism and consequently lower olefin formation and two, by shape selectivity towards lighter products. ZSM-5 zeolite has a unique three-dimensional structure, with much smaller pores compared to the Y-zeolite. This makes ZSM-5 zeolite “shape selective” for cracking the long chain (C6-C10) olefin and paraffin molecules in FCCU. The products of these cracking reactions are predominantly light olefins such as ethylene, propylene and butylene, with a small amount of isobutane. Additionally, transition state shape selectivity effects limit the formation of bulky transition state intermediates inside the pores and avoid the formation of some unwanted reaction products. More than 30% of the world's FCC units are using ZSM-5 additives either continuously or intermittently.

Addition of ZSM-5 to the FCC catalyst enhances the production of light olefins. However, there exists an optimum amount of ZSM-5 beyond which the performance is not enhanced by addition of more ZSM-5. Bulatov and Jirnov analyzed feed conversion over varying concentrations of a component additive containing ZSM-5. ZSM-5 loading was varied from 0 to 40% with a C/O ratio of about 28, a riser outlet temperature of 566° C., a riser partial pressure of 0.0793 MPa, and a contact time of 1.5 s. From the analysis, it was observed that increasing the amount of ZSM-5 levels beyond 10 wt % had only a marginal effect on the yield of propylene as shown in FIG. 1.

The diminishing effectiveness of ZSM-5 at higher loadings was explained by the depletion of the olefin precursors. ZSM-5 generates propylene by selectively cracking olefins in the C5-C9 range. As the loading of ZSM-5 in the catalyst increases, the incremental yield of propylene produced per percentage of ZSM-5 loading decreases.

Similar results were obtained by other researchers. Aitani et al. reported that the addition of 0-20 wt. % ZSM-5 caused an increase in the olefins yield (propylene and butenes) with a corresponding loss in the gasoline yield. Dement'ev et al. studied the conversion of FT liquids into C3-C4 olefins using a commercial Y zeolite-based catalyst and concluded that addition of ZSM-5 increased the ethylene and propylene yields initially with a decrease in butylene yield. Propylene reached a maximum yield (˜20%) at a ZSM-5 loading of 20% and then decreased gradually.

As mentioned earlier, ZSM-5 zeolite has a unique three-dimensional structure with small pores leading to “shape selective” functionality for cracking C6-C10 hydrocarbons to lighter olefins. As is common knowledge, zeolite acidity is higher at lower Si/Al ratios. Changing the Si/Al ratio in ZSM-5 translates to altering the ratio of cracking/isomerization rates. At lower Si/Al ratios, cracking activity is high due to higher acidity but undesirable hydrogen transfer and aromatization reactions also occur leading to increased production of coke and loss of selectivity. Hydrogen transfer reactions in zeolites occur on catalyst surface and are more prominent at low Si/Al ratios when acidity is high. These hydrogen transfer reactions also lead to the production of lower alkanes during hydrocarbon cracking, such as methane, ethane and propane, leading to a drop in the selectivity of light olefins. Konno et al. studied the effect of the Si/Al ratio of ZSM-5 zeolite on the cracking of n-hexane by varying the Si/Al ratio from 50 to 300. As mentioned earlier, zeolites with the lower Si/Al ratios have a higher number of acid sites, enhancing the cracking reaction. However, the lower acidity of the catalyst (higher Si/Al ratio) suppressed undesirable reactions such as consumption of light olefins and formation of BTX. Thus, the highest light olefins yield was obtained at an intermediate Si/Al ratio of 150. The stability of the catalyst is also affected by the Si/Al ratio. It is known that at lower zeolite acidity, i.e. at higher Si/Al ratios, the amount of coke formed is lower due to reduced hydrogen transfer reactions. Thus, increasing the Si/Al ratio extends catalyst lifetimes. Additionally, silico-aluminate zeolites undergo dealumination during regeneration due to steaming at high temperatures. However, zeolites with high Si/Al ratios have lower chances of dealumination and hence are more hydrothermally robust. Thus, from both selectivity and stability point of view, higher Si/Al ratio of zeolites are preferred for maximum light olefin production.

In a heterogeneous catalytic reaction involving medium sized molecules like naphtha and gasoline range hydrocarbons, diffusion of these molecules in and out of catalyst pores usually becomes a rate limiting process. This is especially true of ZSM-5 whose crystal sizes are usually larger than the micropores. Longer diffusion path lengths lead to more time spent on acid sites. A longer residence time enhances the allyl carbenium ions formation by hybrid transfer between the olefin and carbenium ions resulting in BTX and coke formation. which leads to pore-mouth plugging and consequently short catalyst lifetimes 0. Reducing particle size of zeolites is one way of shortening diffusion path lengths and consequently overcome diffusion limitations and thus change the reaction profile diffusion-controlled regime to reaction-controlled regime. Studies have shown that the diffusional resistance of hydrocarbon molecules was about 400 times higher over macro-ZSM-5 crystals when compared to nano-ZSM-5 crystals.

Numerous studies have confirmed the positive effect of lowering crystal size on olefin selectivity and catalyst stability. For instance, Konno et al., studied the effect of zeolite crystal size on the catalyst lifetime by carrying n-hexane cracking over ZSM-5 zeolites with Si/Al=150. Their results are summarized in FIG. 3. From their figure, it can be seen that the initial conversion of n-hexane was almost the same (approximately 94%) for all three crystal sizes (labeled S, M and L for small, medium and large respectively). However, the conversion gradually decreased for the catalyst with large crystal size decreasing to 48% after 50 h while the small and medium sized catalysts maintained high conversions at 82 and 81%, respectively, after 50 h.

Due to coke formation on the catalyst surface, the catalyst has to be frequently regenerated which results in exposure of the catalyst to steam at high temperatures. Under these conditions, the zeolites usually undergo de-alumination which leads to a partial destruction of the framework eventually resulting in irreversible deactivation. Many solutions are proposed to overcome this problem and stabilize the zeolite structure, one of which is phosphorus impregnation.

Addition of phosphorus is supposed to reinforce the zeolite structure and protect it against de-alumination. This way the catalyst retains its acidity and hence its activity during regeneration with the addition of phosphorus. However, addition of phosphorus also caused a decrease in activity by binding with protonic sites, blocking external surface and reducing micropore volume. Hence, the optimal use of phosphorus and its loading on the zeolite depends on the zeolite, the particular application and the Si/Al ratio. For instance, Blasco et al. found that the optimal P/Al molar ratio was 0.5-0.7 for maximum n-decane cracking activity.

In addition to the catalyst properties, the FCC unit's performance depends on operating parameters, including the feed composition, temperature, hydrocarbon partial pressure, residence time, and catalyst-to-oil ratio.

The residence time refers to the time of contact between the oil and the catalyst. For FCC units, reactor residence time depends on the reactor configuration, reaction temperature, and C/O ratio. Generally, for these reactions short contact time is required to prevent unwanted secondary reactions such as hydrogen transfer from occurring [6]. Hence, there is an optimum residence time range, since at lower times the conversion is low and at higher residence times there is loss of selectivity.

Meng et al. studied the effect of residence time in the range between 1.0 s to 3.5 s for the catalytic pyrolysis of Daqing AR over an LCM-5 catalyst at 700° C., CTO of 16, and a steam-to-oil ratio of 0.5. They observed that the yield of total light olefins decreased gradually from about 52.5 wt. % to 49 wt. % by increasing the residence time. Meng et al. studied the effect of contact time on product distribution at 650° C. with a CTO of 17.6 using VGO and CEP-1 catalyst. They varied the residence time between 1.5 s to 4.5 s and found that the feed conversion was relatively constant at about 98.5% in this residence time range. The yields of light olefins first increased slightly until a residence time of about 2.0 s, and then reached a plateau. Similar trends with residence time were observed by Sha et al. and Basua and Kunzru also studied the effect of residence time on propylene yield and report that propylene yield increases slightly and then gradually decreases gradually with increase in residence time.

In conventional FCC units, the reaction temperature is raised by raising the C/O ratio. As expected, increase in temperature will increase the extent of catalytic cracking leading to increase in propylene yields. Propylene and butylene are mainly generated through cracking mechanism via the carbonium ions. However, with further increase in temperature secondary reactions such as cracking and hydrogen transfer occur involving light olefins leading to loss of selectivity.

Meng et al. studied the effect of temperature on feed conversion, selectivity of total light olefins and product distribution in the reaction temperature range of 600-716° C. using a CEP-1 catalyst. They found that as the temperature goes up, the yield of dry gas increases, while that of propylene and butylene decreases due to temperature increase. According to them, the optimum temperature range is 620-660° C. for maximum propylene yield. Several studies show that the yield of ethylene gradually increases with increase in temperature while both propylene and butene go through a maxima and gradually decrease with further increase in temperature.

The amount of catalyst in contact with the feed will depend on the temperature of the regenerated catalyst and the FCC reactor configuration. A large C/O ratio will mean more heat transferred to the feed and hence a higher reaction temperature. This means a higher cracking rate leading to greater feed conversion but also higher thermal cracking along with more unwanted reactions of olefins. Reducing the C/O ratio results in an increased light olefin yield and a decreased dry gas yield. Hence, the C/O ratio has to be optimized according to the specific needs of the process. However, C/O ratios cannot be easily varied in FCC units and are usually limited by the reactor configuration. In conventional FCC units, C/O ratios are usually between 4-10. Meng et al. studied the effect of C/O ratio and concluded that as the C/O ratio goes up, the feed conversion and the yields of dry gas and coke increase, that of gasoline and diesel oil decrease. While propylene yield and overall light olefins yield pass through a maxima, there is little variation in the yields of light olefins with increasing C/O ratio.

Cracking is a unimolecular reaction while hydrogen transfer reaction and oligomerization are bimolecular reactions. Increased oligomerization and hydrogen transfer reactions lead to increased dry gas, aromatics and coke while reducing the light olefins yield. Since a rise in hydrocarbon partial pressure will increase the rate of all bimolecular reactions, lowering hydrocarbon partial pressures is preferred for maximum propylene production. This was confirmed by Hu who studied the effect of hydrocarbon partial pressures on propylene yield and found that raising the hydrocarbon partial pressure increased the amount of dry gas and coke at the expense of gasoline. The change in the rate of hydrogen transfer could also affect the gasoline sulfur concentration and lower the effectiveness of gasoline sulfur reduction catalysts and additives.

Steam is added to FCC units and can act as both a low-cost diluent in the FCC process to reduce the coke deposition on the catalyst and also to improve the dispersion and vaporization of the feed. The amount of steam and the steam-to-oil ratio also affects the product slate. Meng et al. studied the effect of steam-to-hydrocarbon ratio, in the range of 0.2-1.6, for the catalytic pyrolysis of Chinese Daqing atmospheric residue at the reaction temperature of 650° C., the residence time of 2.7 s, and CTO of 15.5. By increasing the steam-to-oil ratio, the yields of dry gas and total light olefins gradually increased while yields of heavier products such as diesel, gasoline, and coke decreased slightly with the conversion remaining largely unchanged. Increasing the steam-to-feed ratio reduces the partial pressure of the hydrocarbons in the feedstock and enhances the cracking of hydrocarbons into the products with low molecular weight. Xiang-Hai et al. reported the same trend for the changes of olefins yield with the steam-to-oil ratio. The catalytic cracking of Daqing oil over an LCM-5 catalyst at 700° C., a CTO of 16, a residence time of 1.9 s, and a steam-to-feed ratio in the range of 0.2-1.0 revealed that the yield of total light olefins increased gradually with increasing the steam-to-oil ratio. However, the steam-to-oil ratio cannot be increased beyond a certain point due to economic and logistic reasons.

The product slate and yield of light olefins is a strong function of the hydrocarbon feedstock properties. During the past decades, the FCC feed mostly consists of heavier hydrocarbons with a growing tendency to incorporate residue. These feeds along with other heavy gas oils and hydrotreated pyrolysis oils with more aromatic contents, are difficult to convert to light olefins. Feeds like tight oils and Fischer-Tropsch waxes would be good candidates as feeds for light olefins production.

Additionally, the production of propylene via cracking requires an excess of hydrogen. Feedstocks that have a high aromatics content are depleted in hydrogen content and do not yield a high amount of olefins under typical FCC operating conditions. Meng et al. studied the effect of feedstock quality on product distribution by investigating four types of feeds using a CEP-1 catalyst at a reaction temperature of 660° C., residence time of 2.2 s, C/O weight ratio of 15.5 and steam-to-oil weight ratio of 0.75. They found that the feed conversion of the four kinds of heavy oils remained very high, above 98%. They also observed that as aromatic content in the feed decreased, the yields of dry gas, diesel oil reduced. The yields of LPG and light olefins increased along with increased coke.

There are many patents and patent application publications that have disclosed various zeolites and modified and promoted zeolites used for catalytic naphtha cracking to produce light olefins. WO 2011162717, US 20070010699, US 2007008280, EP 1117750, US 20070209969 have disclosed the use of metal-modified zeolite, phosphate and water insoluble metal salt prompted porous molecular sieve catalyst, hydrothermally stable molecular sieve catalysts, metal and phosphorus modified zeolites, and alkaline treated zeolite with silica alumina molar ratio of less than 45 for catalytic cracking of naphtha for light olefins production. Similarly, WO 2017/109640 disclosed the use of phosphorus modified HZSM-5 catalysts steam-naphtha cracking to obtain light olefins yield of 45 wt % and higher. U.S. Pat. No. 5,043,552 disclosed various cracking catalysts such as ZSM-5, zeolite A, zeolite X, Zeolite Y, zeolite ZK-5, zeolite ZK-4, synthetic mordenite, dealuminated mordenite, as well as naturally occurring zeolites such as chabazite, faujasite, mordenite, and so on for catalytic cracking process to produce light olefins such as propylene and ethylene. Among these catalysts, the most preferred catalysts mentioned were ammonium and rare earth metal ion exchanged ZSM-5 catalysts.

On the other hand, some patents such as U.S. Pat. Nos. 5,026,935 and 5,026,936 disclosed catalytic cracking of heavier hydrocarbons along with metathesis to produce more light olefins using zeolite catalysts. They have employed zeolites to convert heavier hydrocarbons to light olefins. In this combined process, they have used heavier paraffins in the range of 40 to 95 wt % and heavier olefins in the range of 5 to 60 wt %. EP Patent No. EP 109059B1 preferably used ZSM-5 and ZSM-11 catalysts with silica alumina molar ratio of 400 or less to convert a feed stock containing butenes to dodecenes to propylene. Reaction temperatures of 400 to 600° C. were used for the process. Furthermore, EP 109060B1 disclosed using catalysts consisting of ZSM-5, ZSM-11 having silica alumina molar ratio of 350 or higher, alkaline earth metals and chromium, strontium modified silicalite-1, boralites, silicalites, chromosilcates for the catalytic cracking of butenes to propylene. This process used reaction temperatures of 500 to 600° C. to convert butenes to propylene. WO 2006098712 disclosed using reaction temperatures of 550 to 700° C. for naphtha catalytic cracking to propylene and ethylene, more preferably the reaction temperatures of 650 to 670° C., and catalysts used were zeolites of silica alumina molar ratio of 20 to 200. However, zeolites with higher silica alumina mole ratio were preferred highly due to lower acid sites density to prevent side reaction of propylene and ethylene. The preferred reaction pressure disclosed was in the range of 135 kPa to 450 kPa. Alkaline and titanium promoted ZSM-5 catalysts were used for naphtha cracking to propylene and ethylene production as disclosed in U.S. Pat. No. 10,550,333B2. This patent disclosed that Titanium precursors used were titanium butoxide, titanium tetrachloride, titanium oxychloride, titanium ethoxide, titanium isopropoxide, titanium methoxide, or mixtures thereof.

U.S. Pat. No. 5,043,522 disclosed the process in which paraffinic feedstocks were subjected to very high temperature and low enough pressure over ZSM-5 catalysts to obtain propylene and ethylene. Despite high reaction temperature, the conversion reached only up to 40% and the reactor configuration was not disclosed. Similarly, U.S. Pat. No. 6,222,087 disclosed the use of fluidized bed reactor as well as fixed bed swing reactor for the catalytic cracking of C₄-C₇ paraffins and olefins to propylene and ethylene over ZSM-5 or ZSM-11 zeolite catalysts. The patent shows that due to high oligomerization reaction, high formation of aromatic products such as benzene, toluene, xylene obtained while propylene yield was low. Other patents such as U.S. Pat. Nos. 5,171,921 and 5,043,522 disclosed the use of HZSM-5 and steam activated phosphorus catalysts in catalytic cracking of mixed higher hydrocarbons of paraffins and olefins for the production of light olefins. These patents also disclosed that heat necessary for the fluidized reaction was supplied by regeneration of coked catalysts. U.S. Pat. No. 6,951,968 disclosed the conversion of heavier olefins present in refinery and petrochemical plants to light olefins such as propylene, and ethylene over zeolite catalysts having Si/Al molar ratio of 300 to 1000. Catalytic activity was very low due to very low acid sites density, especially catalysts having higher Si/Al ratio compared to FCC catalysts having much lower silica/alumina ratio. In another patent, U.S. Pat. No. 7,323,099, two independent FCC units were used sequentially to obtain light olefins from gas oil or resid. In this process, heavy oil was processed using large and medium pore zeolite catalysts to convert to naphtha range hydrocarbons, followed by second FCC unit in which naphtha range hydrocarbons converted to light olefins of propylene and ethylene over up to 50% of zeolite catalyst having 0.7 namometers of pore size. The operating condition used for this process involved the temperatures in the range of 500° C. to 650° C. and feed partial pressure in the range of 10 to 40 psia. Publications US 20080035527, US 20060108261, US 200401082745 and WO 2004078881 also disclosed the use of dual or sequential FCC units to cracking heavier hydrocarbons to light olefins of propylene and ethylene using ZSM-5 catalysts.

Some of the results from various patents are summarized in the table below.

TABLE 1
A summary of Naphtha cracking catalysts from patent literature

| | | | Propylene | Ethylene | Temperature, |
|---|---|---|---|---|---|
| Patent # | Feed | Catalyst | yield, wt % | yield, wt % | ° C. |
| WO | Naphtha + | P-modified | 26 | 18 | 650 |
| 2017/109640 | Steam | ZSM5 | | | |
| WO | Naphtha + | ZSM5, | 18 | 17 | 665 |
| 2006/098712 | Steam | Ferrierite, ZSM- | | | |
| | | 22, ZSM-23 | | | |
| U.S. Pat. No. | Naphtha + | ZSM5, | 27 | 18 | 650 |
| 10,550,333B2 | Steam | P/Alkaline/Ti - | | | |
| U.S. Pat. No. | | modified ZSM5 | | | |
| 6,222,087B1 | Butene | P-modified | 42 | 8 | 593 |
| U.S. Pat. No. | | ZSM5 (450) | | | |
| 5,043,522 | Naphtha | HZSM5 + Al2O3 | 17 | 25 | 527 |

### SUMMARY OF INVENTION

Cracking of naphtha using zeolitic catalysts produces significant amounts of ethylene and butenes. These olefins can be converted to produce more propylene using a reaction called metathesis which converts a mixture of 2-butene and ethylene to produce propylene in a reversible reaction. Various supported transition metal oxides have been successfully used as heterogeneous catalysts for metathesis reaction, including molybdenum (Mo), tungsten (W) and rhenium (Re).

Most metathesis catalysts, especially Re-based and Mo-based catalysts, are sensitive to oxygen and/or moisture and, hence, metathesis reactions with these catalysts have to be performed in an inert atmosphere. On the other hand, supported W catalysts have been most successful due to their relatively lower price, better stability, and better resistance to poisoning by impurities. It is generally accepted that a high dispersion of WOx and its interaction with support surface play crucial roles in the catalyst efficiency for the metathesis reaction. For instance, the isolated WOx species is believed to be the active species.

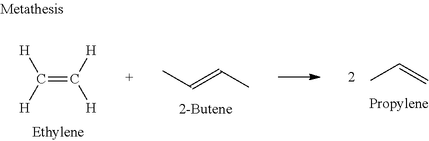

When 1-butene is present in the feed, it has to first be isomerized to 2-butene before it can undergo the metathesis reaction with ethylene. Typically, 1-butene to 2-butene can be catalyzed by silanol groups (Si-OH) present on silica based supports, but adding a second isomerization component such as MgO makes it more effective.

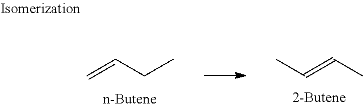

High temperatures promote the isomerization of 2-butene to 1-butene while lower temperatures favor propylene production via metathesis. Hence, it is important to identify the optimal range of temperatures for both reactions to be carried out in the same reactor.

Propylene can also be produced directly from ethylene. This transformation involves a number of sequential reactions such as dimerization (shown below), isomerization and metathesis. For this transformation, various acid microporous catalysts or metal oxide (involving Ni, Re or W) based multifunctional catalysts have been employed.

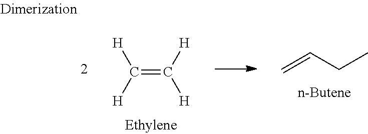

We have recently discovered that the addition of small amounts of alkaline earth metal oxides (CaO, MgO) as the isomerization function and transition metal oxides (such as Group VI metal oxides (MoO₃, WO₃)) as the metathesis function significantly enhances the production of propylene using Zeolites.

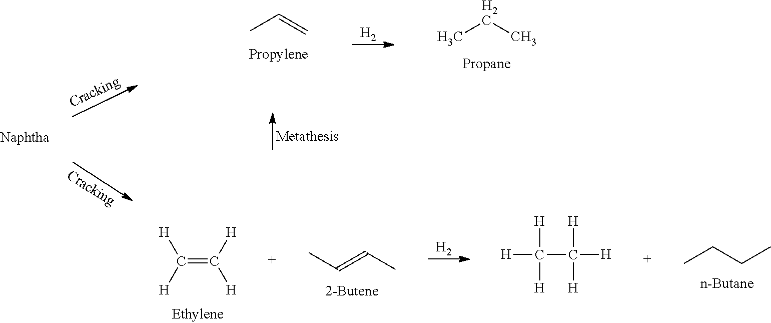

As shown in the reaction scheme above, once the light olefins are formed by cracking on the acid sites of the zeolite, the 1-butene molecules undergo isomerization catalyzed by alkali earth metal oxides and then react with ethylene to produce more propylene.

In a first aspect, the invention provides a cracking catalyst suitable for the selective cracking of hydrocarbons to light olefins ethylene and propylene with a composition of the general formula (Zeolite)(IF)(MF), wherein:
a) Zeolite represents zeolites from the family of Pentasils, Faujasites, Beta and Mordenite, or mixtures thereof,
b) IF, the Isomerization function, represents oxides of Alkaline Earth metals selected from the group 2 elements or mixtures thereof,
c) MF, the metathesis function, represents oxides of transition metals or mixtures thereof; and
characterizable by conversion ≥85% or >95%, propylene selectivity ≥30 wt % or >40 wt % and a Propylene/Ethylene Ratio >1 wt/wt or >2 wt/wt using a test where the catalyst is loaded in a fixed-bed reactor such that the 50>d_{T}/d_{P}>10 (diameter of tube to diameter of catalyst particles) and 200>L/d_{P}>50 (length of catalyst bed to diameter of catalyst particles) and 2>d_{P}>0.5 mm exposed to a feed stream of 1-hexene at a temperature of 625° C., atmospheric pressure and a feed rate of 60 hr⁻¹ weight hourly space velocity. Alternatively, in the above characterization test, the Propylene/Ethylene Ratio is in the range of 1 to 5, or 1 to 4, or 1 to 3; and propylene selectivity in the range of 30-70, 30-60, or 30-50 wt %. Preferably, the transition metals are one or more of Cr, Mo, and W.

Since the catalyst cannot be completely distinguished from the prior art based solely on its elemental composition, the measurement described above is needed for a unique characterization of the catalyst. In any of its aspects, the catalyst may be further characterized by any of the compositions or physical characteristics described herein.

In another aspect, the invention provides a method of synthesizing the cracking catalyst using the incipient wetness method. A Zeolite such as Y, USY, ZSM-5 is impregnated with salt solutions of isomerization and metathesis metals, then dried and finally calcined to produce the final form of the catalyst. In a related aspect, the invention provides a method of making the catalyst, comprising: i) dissolving appropriate salts of the Isomerization and Metathesis function or mixtures thereof in water; ii) impregnating the Zeolites with the salt solution; iii) drying the Zeolite impregnated with salt solutions; iv) adding the desired binder to the impregnated Zeolite; and v) calcining the resultant mixture for 2-6 hrs in an oxygen containing atmosphere, preferably air to produce the cracking catalyst particle. The invention includes catalysts made by any of the methods described herein.

A binder such as alumina, silica or titania may be added to the catalyst, then calcined to form the final particle wherein the BET surface area >100 m²/g and wherein diameter of the catalyst particle is between 30-3000 microns.

Another aspect of the invention provides a continuous method for cracking hydrocarbons with a suitable cracking catalyst having 4-100 carbon atoms wherein the process is performed at a reaction temperature of 500-800° C., a weight hourly space velocity of 1 -100 hr⁻¹ a pressure of 0.01-0.2 MPa. The hydrocarbon feedstock is contacted with the catalyst under cracking conditions for a reaction period in the range of about 0.05 second to 10 minutes. In the method, at least 55 wt % of the hydrocarbons in the mixture of hydrocarbons are converted with a propylene selectivity of at least 30 wt %, and a propylene to ethylene mass ratio of at least 1. Following the reaction period, the catalyst may thereafter be regenerated by contacting the catalyst with air. The catalyst regeneration can be performed at a reaction temperature of 500-800° C., a pressure of 0.01-0.2 MPa and a regeneration period ranging from about 1 to 10 minutes. The process can be carried out in a fluidized bed reactor or a fixed-bed swing reactor.

The invention is further illustrated in the examples below. In some preferred embodiments, the invention may be further characterized by any selected descriptions from the examples, for example, within ±20% (or within ±10%) of any of the values in any of the examples, tables or figures; however, the scope of the present invention, in its broader aspects, is not intended to be limited by these examples.

In some preferred embodiments, the invention provides advantages such as: the product of the catalyst activity and catalyst selectivity exceeding 24 ton of propylene per hour per ton of catalyst; and the overall catalyst consumption does not exceed 1 kg of catalyst per ton of product. None of the prior art catalysts listed in the prior art meet these characteristics simultaneously.

### GLOSSARY

Calcination Temperature—The term “calcination temperature” refers to the maximum temperature utilized as an intermediate step in the catalyst synthesis procedure intended to convert the metal salts to their oxide form.

Regeneration Temperature—The catalyst may be regenerated under flowing air gas at elevated temperatures in order to remove heavier hydrocarbons (coke) from the active catalyst structure. The maximum temperature used in this step is referred to as the “regeneration temperature.”

Conversion—The term “conversion of a reactant” refers to the reactant mole or mass change between a material flowing into a reactor and a material flowing out of the reactor divided by the moles or mass of reactant in the material flowing into the reactor.

Pore size—Pore size relates to the size of a molecule or atom that can penetrate into the pores of a material. As used herein, the term “pore size” for zeolites and similar catalyst compositions refers to the Norman radii adjusted pore size well known to those skilled in the art. Determination of Norman radii adjusted pore size is described, for example, in Cook, M.; Conner, W. C., “How big are the pores of zeolites?” Proceedings of the International Zeolite Conference, 12th, Baltimore, Jul. 5-10, 1998; (1999), 1, pp 409-414.

One of ordinary skill in the art will understand how to determine the pore size (e.g., minimum pore size, average of minimum pore sizes) in a catalyst. For example, x-ray diffraction (XRD) can be used to determine atomic coordinates. XRD techniques for the determination of pore size are described, for example, in Pecharsky, V. K. et at, “Fundamentals of Powder Diffraction and Structural Characterization of Materials,” Springer Science+Business Media, Inc., New York, 2005. Other techniques that may be useful in determining pore sizes (e.g., zeolite pore sizes) include, for example, helium pycnometry or low-pressure argon adsorption techniques. These and other techniques are described in Magee, J. S. et at, “Fluid Catalytic Cracking: Science and Technology,” Elsevier Publishing Company, Jul. 1, 1993, pp. 185-195. Pore sizes of mesoporous catalysts may be determined using, for example, nitrogen adsorption techniques, as described in Gregg, S. J. at al, “Adsorption, Surface Area and Porosity,” 2nd Ed., Academic Press Inc., New York, 1982 and Rouquerol, F. et al, “Adsorption by powders and porous materials. Principles, Methodology and Applications,” Academic Press Inc., New York, 1998.

“Particle size” is the number average particle size, and, for non-spherical particles, is based on the largest dimension.

Residence Time—Residence time is the time a substance is in the reaction vessel. It can be defined as the volume of the reactor divided by the flow rate (by volume per second) of gases into the reactor.

Selectivity—The term “selectivity” refers to the amount of production of a particular product (or products) as a percent of all products resulting from a reaction. For example, if 100 grams of products are produced in a reaction and 80 grams of olefins are found in these products, the selectivity to olefins amongst all products is 80/100=80%. Selectivity can be calculated on a mass basis, as in the aforementioned example, or it can be calculated on a molar basis, where the selectivity is calculated by dividing the moles of a particular product by the moles of all products. Unless specified otherwise, selectivity is on a mass basis.

Yield—The term “yield” is used herein to refer to the amount of a product flowing out of a reactor divided by the amount of reactant flowing into the reactor, usually expressed as a percentage or fraction. Mass yield is the mass of a particular product divided by the weight of feed used to prepare that product. When unspecified, “%” refers to mass % which is synonymous with weight %. Ideal gas behavior is assumed so that mole % is the same as volume % in the gas phase.

As is standard patent terminology, the term “comprising” means “including” and does not exclude additional components. Any of the inventive aspects described in conjunction with the term “comprising” also include narrower embodiments in which the term “comprising” is replaced by the narrower terms “consisting essentially of” or “consisting of.” As used in this specification, the terms “includes” or “including” should not be read as limiting the invention but, rather, listing exemplary components. As is standard terminology, “systems” include to apparatus and materials (such as reactants and products) and conditions within the apparatus. All ranges are inclusive and combinable. For example, when a range of “1 to 5’ is recited, the recited range should be construed as including ranges “1 to 4”, “1 to 3”, “1-2”, “1-2 & 4-5”, “1-3 & 5”, “2-5”, any of 1, 2, 3, 4, or 5 individually, and the like.

### BRIEF DESCRIPTION OF THE DRAWINGS

FIG. 1 shows performance of various catalysts for naphtha cracking. Cat A is the as-is zeolite. Cat-B is the sample with the zeolite and binder. Cat-C is the zeolite+binder+isomerization function, while Cat D contains the zeolite+binder+isomerization function+metathesis function. The results provide quantifiable advantages of the cracking catalyst invention.

FIG. 2 shows the effect of SAR (Si/Al ratio) on performance of ZSM-5 based catalysts for naphtha cracking.

### EXAMPLES

### Example 1

The catalyst used was a commercial zeolite ZSM-5 from Zeolyst having a SiO₂/Al₂O₃ molar ratio of 30 (Si/Al of 15) and a sodium content of 0.05% by weight. The zeolite was pelletized and sieved to 0.5-1.0 mm particles.

This catalyst is designated as Catalyst A

### Example 2

The catalyst was prepared by dispersing alumina binder in the form of acidic Dispal (T25N4-80) from Sasol in DI water for 30 minutes, followed by mixing ZSM-5 zeolite having a SiO₂/Al₂O₃ molar ratio of 30 (Si/Al of 15) to the dispersed Dispal for 30 minutes. The excess water was evaporated by heating to obtain paste form. The paste was dried overnight at 120° C., followed by calcination at 500° C. for 4 hours. The alumina binder content in the mixture was targeted to be 60 wt %.

This catalyst is designated as Catalyst B

### Example 3

The catalyst was prepared by dispersing alumina binder in the form of acidic Dispal (T25N4-80) from Sasol in DI water for 30 minutes, followed by mixing ZSM-5 zeolite having a SiO₂/Al₂O₃ molar ratio of 30 (Si/Al of 15) to the dispersed Dispal for 30 minutes. The excess water was evaporated by heating to obtain paste form. The paste was dried overnight at 120° C. After drying, the catalyst was ground and a solution of 4 wt % magnesium oxide in the form of Mg(NO₃)₂*6H₂O precursor in DI water was added to dried catalyst drop-wise via incipient wetness technique followed by drying and calcination at 500° C. for 4 hours.

This catalyst is designated as Catalyst C.

### Example 4

The catalyst was prepared by dispersing alumina binder in the form of acidic Dispal (T25N4-80) from Sasol in DI water for 30 minutes, followed by mixing ZSM-5 zeolite having a SiO₂/Al₂O₃ molar ratio of 30 (Si/Al of 15) to the dispersed Dispal for 30 minutes. The excess water was evaporated by heating to obtain paste form. The paste was dried overnight at 120° C. After drying, the catalyst was ground and a mixed solution of 4 wt % magnesium oxide in the form of Mg(NO₃)₂*6H₂O precursor and 5 wt % tungsten oxide in the form of (NH₄)₆W₁₂O₃₉*H₂O in DI water was added to dried catalyst drop-wise via incipient wetness technique followed by drying and calcination at 500° C. for 4 hours.

This catalyst is designated as Catalyst D.

### Example: Catalyst Tests

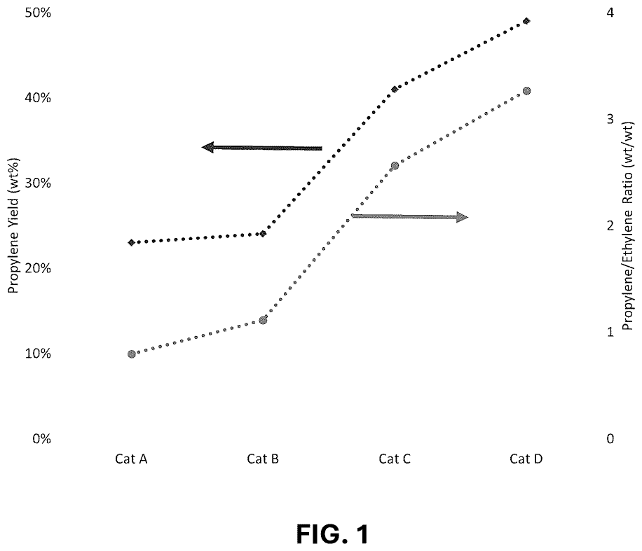

The above catalysts were tested as follows: a synthesized catalyst was loaded in a fixed-bed quartz reactor with a diameter of 10 mm such that d_{T}/dₚ>10 and L/dₚ>50. The catalyst was activated and steamed at 725° C. for 16 hours. After steaming, the reactor temperature was lowered to reaction temperature of 625° C. The reaction was conducted at atmospheric pressure and 60/h WHSV. Product samples were withdrawn and analyzed using gas chromatographs equipped with Plot-Q 30 m columns. The results of naphtha cracking over the above catalysts are shown in FIG. 1.

### Example 5

The catalyst was prepared by dispersing alumina binder in the form of acidic Dispal (T25N4-80) from Sasol in DI water for 30 minutes, followed by mixing ZSM-5 zeolite having a SiO₂/Al₂O₃ molar ratio of 23 (Si/Al of 11.5) to the dispersed Dispal for 30 minutes. The excess water was evaporated by heating to obtain paste form. The paste was dried overnight at 120° C. After drying, the catalyst was ground and a mixed solution of 9 wt % magnesium oxide in the form of Mg(NO₃)₂*6H₂O precursor and 1 wt % tungsten oxide in the form of (NH₄)₆W₁₂O₃₉*H₂O in DI water was added to dried catalyst drop-wise via incipient wetness technique followed by drying and calcination at 500° C. for 4 hours. The alumina binder content in the mixture was targeted to be 60 wt %.

This catalyst is designated as Catalyst E.

### Example 6

The catalyst used was as in Example 5, with the only difference being the SiO₂/Al₂O₃ molar ratio was 30 (Si/Al of 15) instead of 23.

This catalyst is designated as Catalyst F.

### Example 7

The catalyst used was as in Example 5, with the only difference being the SiO₂/Al₂O₃ molar ratio was 80 (Si/Al of 40) instead of 23.

This catalyst is designated as Catalyst G.

### Example 8

The catalyst used was as in Example 5, with the only difference being the SiO₂/Al₂O₃ molar ratio was 280 (Si/Al of 140) instead of 23.

This catalyst is designated as Catalyst H.

### Example 9

The catalyst used was as in Example 5, with the only difference being the SiO₂/Al₂O₃ molar ratio was 371 (Si/Al of 185.5) instead of 23.

This catalyst is designated as Catalyst I.

### Catalyst Tests

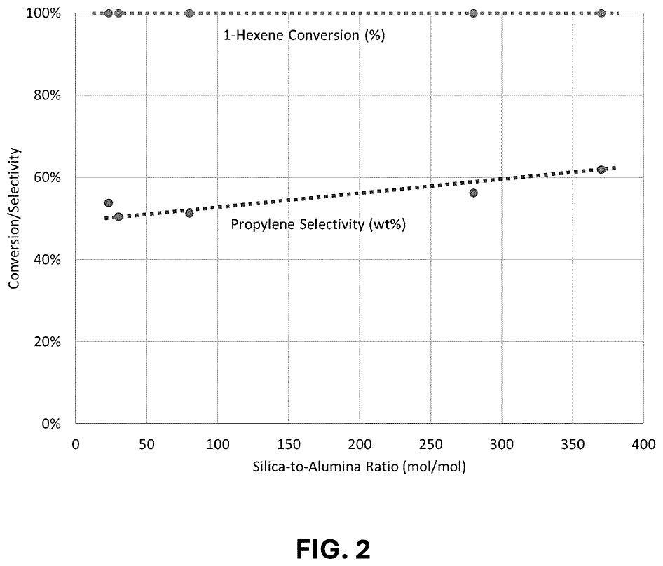

The above catalysts were tested as in Example Catalyst tests. The results of naphtha cracking over Catalysts E through I are shown in FIG. 2. Based on these results, ZSM-5 zeolites with higher SAR ratios produce higher amounts of propylene.

### Example 10

The catalyst used was as in Example 5, with the only difference being the zeolite used was H-Y zeolite having a SiO₂/Al₂O₃ molar ratio of 5.2 instead of ZSM5 zeolite.

This catalyst is designated as Catalyst J

### Example 11

The catalyst used was as in Example 5, with the only difference being the zeolite used was H-Beta zeolite having a SiO₂/Al₂O₃ molar ratio of 25 instead of ZSM5 zeolite.

This catalyst is designated as Catalyst K.

### Catalyst Tests

The above catalysts were tested as in Example Catalyst tests. The results of naphtha cracking over the above catalysts are shown in Table 2.

TABLE 2
Performance of various catalysts for naphtha cracking

| Zeolite Used | H-Y | H-Beta |
|---|---|---|
| 1-Hexene Conversion | 100% | 100% |
| Propylene Selectivity | 44% | 55% |

Results in Table 2 clearly show merits of the invention whereby adding an isomerization and metathesis function to conventional zeolites is able to enhance cracking rates and enhance selectivity to propylene production.

### REFERENCES

1. Lawler D, Letzsch W, Dhaidan F (2005) Advances in fccu technology for the production of olefins. 25-29 Sep. 2005
2. Nexant (2009) Propylene technology: the next generation
3. Ren T, Patel M, Blok K (2006) Olefins from conventional and heavy feedstocks: energy use in steam cracking and alternative processes. Energy Fuels 31:425-451
4. Perego C, Millini R (2013) Porous materials in catalysis: challenges for mesoporous materials. Chem Cracking catalyst Rev 42:3956-3976.
5. Von Ballmoos R, Hayward C-MT (1991) Matrix vs zeolite contributions to the acidity of fluid cracking catalysts. In: Öhlmann G, Fricke R (eds) Studies in surface science and catalysis, vol 65. Elsevier, pp 171-183.
6. Yan HT, Mao RLV (2010) Hybrid catalysts used in the Catalytic Steam Cracking process (CSC): influence of the pore characteristics and the surface acidity properties of the ZSM-5 zeolite-based component on the overall catalytic performance. Appl Catal A Gen 375:63-69
7. Zhao X, Roberie TG (1999) ZSM-5 additive in fluid catalytic cracking. 1 Effect of additive level and temperature on light olefins and gasoline olefins. Ind Eng Chem Res 38:3847-3853.
8. Degnan TF, Chitnis GK, Schipper PH (2000) History of ZSM-5 fluid catalytic cracking additive development at Mobil. Microporous Mesoporous Mater 35-36:245-252.
9. Arandes JM, Torre I, Azkoiti MJ, Ereña J, Olazar M, Bilbao J (2009) HZSM-5 zeolite as catalyst additive for residue cracking under FCC conditions. Energy Fuels 23:4215-4223.
10. Bulatov RM, Jirnov BS (2009) FCC process of heavy feed stock with improved yield of light olefins Oil and Gas Business. http://wwwogbusru/eng/authors/Bulatov/Bulatov_1pdf
11. Radcliffe C (2007) The FCC unit as a propylene source catalysts additives. http://wwwdigitalrefiningcom/article_1000312pdf 14
12. den Hollander MA, Wissink M, Makkee M, Moulijn JA (2002) Gasoline conversion: reactivity towards cracking with equilibrated FCC and ZSM-5 catalysts. Appl Catal A Gen 223:85-102.
13. Bari Siddiqui MA, Aitani AM, Saeed MR, Al-Khattaf S (2010) Enhancing the production of light olefins by catalytic cracking of FCC naphtha over mesoporous ZSM-5 catalyst. Topics Catal 53:1387-1393.
14. Möller KP, Böhringer W, Schnitzler AE, van Steen E, O'Connor CT (1999) The use of a jet loop reactor to study the effect of crystal size and the co-feeding of olefins and water on the conversion of methanol over HZSM-5. Microporous Mesoporous Mater 29:127-144.
15. Nishiyama N, Kawaguchi M, Hirota Y, Van Vu D, Egashira Y, Ueyama K (2009) Size control of SAPO-34 crystals and their catalyst lifetime in the methanol-to-olefin reaction. Appl Catal A Gen 362:193-199.
16. Ding L, Zheng Y, Hong Y, Ring Z (2007) Effect of particle size on the hydrothermal stability of zeolite beta. Microporous Mesoporous Mater 101:432-439.
17. Firoozi M, Baghalha M, Asadi M (2009) The effect of micro and nano particle sizes of H-ZSM-5 on the selectivity of MTP reaction. Catal Comm 10:1582-1585.
18. Konno H, Okamura T, Kawahara T, Nakasaka Y, Tago T, Masuda T (2012) Kinetics of n-hexane cracking over ZSM-5 zeolites-effect of crystal size on effectiveness factor and catalyst lifetime. Chem Eng J 207-208:490-496.
19. Konno H, Okamura T, Nakasaka Y, Tago T, Masuda T (2012) Effects of crystal size and Si/Al ratio of MFI-type zeolite catalyst on n-hexane cracking for light olefin synthesis. J Jpn Petrol Inst 55:267-274
20. Mochizuki H, Yokoi T, Imai H, Watanabe R, Namba S, Kondo JN, Tatsumi T (2011) Facile control of crystallite size of ZSM-5 catalyst for cracking of hexane. Microporous Mesoporous Mater 145:165-171.
21. Corma A, Mengual J, Miguel PJ (2012) Stabilization of ZSM-5 zeolite catalysts for steam catalytic cracking of naphtha for production of propene and ethene. Appl Catal A Gen 421-422:121-134
22. Corma A, Mengual J, Miguel PJ (2013) IM-5 zeolite for steam catalytic cracking of naphtha to produce propene and ethene. An alternative to ZSM-5 zeolite. Appl Catal A 106-115:460-461
23. Gao X, Tang Z, Ji D, Zhang H (2009) Modification of ZSM-5 zeolite for maximizing propylene in fluid catalytic cracking reaction. Catal Comm 10:1787-1790
24. Guisnet M, Ribeiro FR (2011) Catalytic Science Series. Deactivation and regeneration of zeolite catalysts, vol 9. Imperial College Press, London
25. Teimouri Sendesi SM, Towfighi J, Keyvanloo K (2012) The effect of Fe, P and Si/Al molar ratio on stability of HZSM-5 catalyst in naphtha thermal-catalytic cracking to light olefins. Catal Comm 27:114-118.
26. Zhao G, Teng J, Xie Z, Jin W, Yang W, Chen Q, Tang Y (2007) Effect of phosphorus on HZSM-5 catalyst for C4-olefin cracking reactions to produce propylene. J Catal 248:29-37.
27. Cerqueira HS, Caeiro G, Costa L, Ramôa Ribeiro F (2008) Deactivation of FCC catalysts. J Mol Catal A: Chem 292:1-13
28. Blasco T, Corma A, Martínez-Triguero J (2006) Hydrothermal stabilization of ZSM-5 catalytic-cracking additives by phosphorus addition. J Catal 237:267-277.
29. Vogt, E.T.C .; Weckhuysen, B.M. Fluid catalytic cracking: Recent developments on the grand old lady of zeolite catalysis. Chem. Cracking catalyst. Rev.2015, 44, 7342-7370.
30. Yoshimura, Y.; Kijima, N.; Hayakawa, T.; Murata, K.; Suzuki, K.; Mizukami, F.; Matano, K.; Konishi, T.; Oikawa, T.; Saito, M.; et al. Catalytic cracking of naphtha to light olefins. Catal. Surv. Jpn. 2001, 4, 157-167.
31. Banjare, M.; Pal, M.P.; Nanacharaiah, C.; Desai, V.S.; Guria, S.; Vardhan, H.; Mondal, B.C. Use of phased array ultrasonic testing for sizing of hydrogen blisters in LPG wash water vessel in INDMAX unit. Int. J. Mech. Ind. Eng. 2012, 1, 203-207.
32. Parthasarathi, R.S.; Alabduljabbar, S.S. HS-FCC high-severity fluidized catalytic cracking: A newcomer to the FCC family. Appl. Petrochem. Res. 2014, 4, 441-444.
33. Haribal, V.P.; Chen, Y.; Neal, L.; Li, F. Intensification of ethylene production from naphtha via a redox oxy-cracking scheme: Process simulations and analysis. Engineering 2018, 4, 714-721.
34. Konno, H.; Ohnaka, R.; Nishimura, J.-I.; Tago, T.; Nakasaka, Y.; Masuda, T. Kinetics of the catalytic cracking of naphtha over ZSM-5 zeolite: Effect of reduced crystal size on the reaction of naphthenes. Catal. Sci. Technol. 2014, 4, 4265-4273.
35. Xu, S.; Zhang, X.; Cheng, D.-G.; Chen, F.; Ren, X. Effect of hierarchical ZSM-5 zeolite crystal size on diffusion and catalytic performance of n-heptane cracking. Front. Chem. Sci. Eng. 2018, 12, 780-789.
36. Mochizuki, H.; Yokoi, T.; Imai, H.; Watanabe, R.; Namba, S.; Kondo, J.N.; Tatsumi, T. Facile control of crystallite size of ZSM-5 catalyst for cracking of hexane. Microporous Mesoporous Mater. 2011, 145, 165-171.
37. Rownaghi, A.A.; Rezaei, F.; Hedlund, J. Selective formation of light olefin by n-hexane cracking over HZSM-5: Influence of crystal size and acid sites of nano- and micrometer-sized crystals. Chem. Eng. J. 2012, 191, 528-533.
38. Konno, H.; Tago, T.; Nakasaka, Y.; Ohnaka, R.; Nishimura, J.-I.; Masuda, T. Effectiveness of nano-scale ZSM-5 zeolite and its deactivation mechanism on catalytic cracking of representative hydrocarbons of naphtha. Microporous Mesoporous Mater. 2013, 175, 25-33.
39. Aitani, A. Maximization of FCC light olefins by high severity operation and ZSM-5 addition. Catal. Today 2000, 60, 111-117.
40. Dement'Ev, K.I.; Sagaradze, A.D.; Kuznetsov, P.S.; Palankoev, T.A.; Maximov, A.L. Selective production of light olefins from Fischer-Tropsch synthetic oil by catalytic cracking. Ind. Eng. Chem. Res. 2020, 59, 15875-15883.
41. Bellussi, G.; Pollesel, P. Industrial applications of zeolite catalysis: Production and uses of light olefins. In Studies in Surface Science and Catalysis; Elsevier: Amsterdam, The Netherlands, 2005; Volume 158, pp. 1201-1212.
42. The changing dynamics of olefin supply/demand. Catal. Today 2005, 106, 10-14.
43. Zhu, X.; Liu, S.; Song, Y.; Xu, L. Catalytic cracking of C₄ alkenes to propene and ethene: Influences of zeolites pore structures and Si/Al₂ ratios. Appl. Catal. A Gen. 2005, 288, 134-142.
44. Meng X, Xu C, Gao J, Li L (2005) Studies on catalytic pyrolysis of heavy oils: reaction behaviors and mechanistic pathways. Appl Catal A General 294:168-176.
45. Hu R, Weatherbee G, Ma H, Roberie T, Cheng W-C (2008) Effect of hydrocarbon partial pressure on FCC propylene. Catal Addit Fluid Catal Crack Propylene Maximisation 139:1-10
46. Sha, Y.; Cui, Z.; Wang, M.; Wang, G.; Zhang, J. Olefin production by heavy-oil contact cracking. Pet. Technol. 1999, 28, 618-621.
47. Basu, B.; Kunzru, D. Catalytic pyrolysis of naphtha. Ind. Eng. Chem. Res. 1992, 31, 146-155.
48. Meng, X.-H.; Xu, C.-M.; Li, L.; Gao, J.-S. Studies on the kinetics of heavy oil catalytic pyrolysis. Ind. Eng. Chem. Res. 2003, 42, 6012-6019.
49. Tian, Y.; Che, Y.; Chen, M.; Feng, W.; Zhang, J.; Qiao, Y. Catalytic upgrading of vacuum residue-derived cracking gas-oil for maximum light olefin production in a combination of a fluidized bed and fixed bed reactor. Energy Fuels 2019, 33, 7297-7304.
50. Al-Khattaf, S.S.F.; Saeed, M.R.; Aitani, A.; Klein, M.T. Catalytic cracking of light crude oil to light olefins and naphtha over E-Cat and MFI: Microactivity test versus advanced cracking evaluation and the effect of high reaction temperature. Energy Fuels 2018, 32, 6189-6199.
51. T. Hahn, E. V. Kondratenko and D. Linke, Chem. Commun., 2014, 50, 9060 -9063.
52. X. J. Li, W. P. Zhang, S. L. Liu, S. J. Xie, X. X. Zhu, X. H. Bao and L. Y. Xu, J. Mol. Catal. A: Chem., 2009, 313, 38 -43.
53. R. Westhoff and J. A. Moulijn, J. Catal., 1977, 46, 414 -416.
54. E. Mazoyer, K. C. Szeto, N. Merle, S. Norsic, O. Boyron, J. M. Basset, M. Taoufik and C. P. Nicholas, J. Catal., 2013, 301, 1-7.
55. S. J. Huang, S. L. Liu, W. J. Xin, J. Bai, S. J. Xie, Q. X. Wang and L. Y. Xu, J. Mol. Catal. A: Chem., 2005, 226, 61-68.
56. C. Meetings Chemistry, 2003, 9, 971-975.
57. S. Lwin and I. E. Wachs, ACS Catal., 2015, 6, 272-278.
58. S. Lwin and I. E. Wachs, ACS Catal., 2014, 4, 2505-2520.
59. N. Gholampour, M. Yusubov and F. Verpoort, Catal. Rev., 2016, 58, 113-156.
60. Wang, Changji and Jiang, Feng and Zuo, Guangzheng and Liu, Bing and Li, Hanxu and Liu, Xiaohao., RSC Adv., 2019,9, 40618-40627.
61. Hua, D.; Zhou, Z.; Hua, Q.; Li, J.; Lu, X.; Xie, Y.; Xiao, H.; Li, M.; Yang, J. Transformation of 2-Butene into Propene on WO₃/MCM-48: Metathesis and Isomerization of n-Butene. Catalysts 2018, 8, 585.
62. Lin, B.; Zhang, Q.; Wang, Y. Catalytic Conversion of Ethylene to Propylene and Butenes over H-ZSM-5. Ind. Eng. Chem. Res. 2009, 48, 10788-10795.
63. Zhou, H.; Wang, Y.; Wei, F.; Wang, D.; Wang, Z. Kinetics of the Reactions of the Light Alkenes over SAPO-34. Appl. Catal., A 2008, 348, 135-141.
64. Oikawa, H.; Shibata, Y.; Inazu, K.; Iwase, Y.; Murai, K.; Hyodo, S.; Kobayashi, G.; Baba, T. Highly Selective Conversion of Ethene to Propene over SAPO-34 as a Solid Acid Catalyst. Appl. Catal., A 2006, 312, 181-185.
65. Iwamoto, M.; Kosugi, Y. Highly Selective Conversion of Ethylene to Propene and Butenes on Butenes on Nickel Ion-Loaded Mesoporous Silica Catalysts. J. Phys. Chem. C 2007, 111, 13-15.
66. Perea, L. A.; Wolff, T.; Veit, P.; Hilfert, L.; Edelmann, F. T.; Hamel, C.; Seidel-Morgenstern, A. Alumino-Mesostructured Ni Catalyst for the Direct Conversion of Ethene to Propene. J. Catal. 2013, 305, 154-168.
67. Frey, A.; Hinrichsen, O. Comparison of Differently Synthesis Ni(Al)MCM-48 Catalysts in the Ethene to Propene Reaction. Microporous Mesoporous Mater. 2012, 164, 164-171.
68. Taoufik, M.; Le Roux, E.; Thivolle-Cazat, J.; Basset, J. M. Direct Transformation of Ethylene into Propylene Catalyzed by a Tungsten Hydride Supported on Alumina: Trifunctional Single-Site Catalysis. Angew. Chem., Int. Ed. 2007, 46, 7202-7205.
69. Li, L .; Palcheva, R. D .; Jens, K. J. Conversion of Ethene to Propene by a Dual Function NiSO₄/Re₂O₇/γ-Al₂O₃ Catalyst. Top. Catal. 2013, 56, 783-788.

1. A cracking catalyst suitable for the selective cracking of hydrocarbons to light olefins ethylene and propylene with a composition of the general formula (Zeolite)(IF)(MF), wherein:
a) Zeolite represents zeolites from the family of Pentasils, Faujasites, Beta and Mordenite, or mixtures thereof wherein the Zeolite species makes up 1 to 75 wt % of the total weight of the cracking catalyst;
b) Isomerization function (IF) represents oxides of Alkaline Earth metals selected from the group 2 elements or mixtures thereof wherein the Isomerization Function (IF) makes up 1 to 25 wt % of the total weight of the cracking catalyst;
c) metathesis function (MF) represents oxides of Transition metals or mixtures thereof wherein Metathesis Function (MF) makes up 1 to 25 wt % of the total weight of the cracking catalyst; and
characterizable by conversion ≥85%, propylene selectivity ≥30 wt % and a Propylene/Ethylene Ratio ≥1 wt/wt using a test where the catalyst is loaded in a fixed-bed reactor such that the 50≥d_{T}r/d_{P}≥10 (diameter of tube to diameter of catalyst particles) and 200≥L/d_{P}≥50 (length of catalyst bed to diameter of catalyst particles) and 2≥d_{P}≥0.5 mm (particle diameter) exposed to a feed stream of 1-hexene at a temperature of 625° C., atmospheric pressure and a feed rate of 60 hr⁻¹ weight hourly space velocity.

2. The cracking catalyst according to claim 1 wherein the Zeolite species makes up 1 to 50 wt % of the total weight of the cracking catalyst.

3. The cracking catalyst according to claim 1 wherein the Zeolite species makes up 1 to 40 wt % of the total weight of the cracking catalyst.

4. The cracking catalyst according to claim 1 wherein the wherein the crystalline zeolite is selected from the group consisting of ZSM-5, Zeolite-Beta, Mordenite, Zeolite X and Zeolite Y and mixtures thereof.

5. The cracking catalyst according to claim 1 wherein the Silica-to-Alumina molar ratio of the Zeolite species varies from 2-1000.

6. The cracking catalyst according to claim 1 wherein the Silica-to-Alumina molar ratio of the Zeolite species varies from 2-400.

7. The cracking catalyst according to claim 1 wherein the Isomerization Function (IF) is selected from the group consisting of Beryllium (Be), Magnesium (Mg), Calcium (Ca), Strontium (Sr), Barium (Ba) and mixtures thereof.

8. The cracking catalyst according to claim 1 wherein the Isomerization Function (IF) makes up 1 to 15 wt % of the total weight of the cracking catalyst.

9. The cracking catalyst according to claim 1 wherein the Isomerization Function (IF) makes up 1 to 10 wt % of the total weight of the cracking catalyst.

10. The cracking catalyst according to claim 1 wherein Metathesis Function (MF) is selected from the group consisting of Titanium, Chromium, Manganese, Iron, Cobalt, Nickel, Copper, Zinc, Yttrium, Zirconium, Niobium, Molybdenum, Tungsten, Lanthanum, Cerium and mixtures thereof.

11. The cracking catalyst according to claim 1 wherein Metathesis Function (MF) makes up 1 to 15 wt % of the total weight of the cracking catalyst.

12. (canceled)

13. The cracking catalyst according to claim 1 wherein mass ratio of the Isomerization Function (IF) to the Metathesis Function (MF) varies from 10:1 to 1:1.

14. (canceled)

15. The cracking catalyst according to claim 1 wherein a support makes up 1 to 25 wt % of the total weight of the cracking catalyst.

16. The cracking catalyst according to claim 1 wherein a support comprises of silica, alumina, clays, kaolin, bentonite, attapulgite, or mixtures thereof.

17. (canceled)

18. The cracking catalyst according to claim 1 wherein the BET surface area >100 m²/g.

19. The cracking catalyst according to claim 1 wherein the Transition metals are group 6 transition metals.

20. The cracking catalyst according to claim 1 characterizable by conversion >95%, propylene selectivity >40 wt % and a Propylene/Ethylene Ratio >2 wt/wt using a test where the catalyst is loaded in a fixed-bed reactor such that the 50≥d_{T}/d_{P}≥10 (diameter of tube to diameter of catalyst particles) and 200÷L/d_{P}≥50 (length of catalyst bed to diameter of catalyst particles) and 2≥d_{P}≥0.5 mm (particle diameter) exposed to a feed stream of 1-hexene at a temperature of 625° C., atmospheric pressure and a feed rate of 60 hr⁻¹ weight hourly space velocity.

21. A method of making the catalyst of claim 1 comprising the steps of:
i) dissolving appropriate salts of the Isomerization and Metathesis function or mixtures thereof in water;
ii) impregnating the Zeolites with the salt solution;
iii) drying the Zeolite impregnated with salt solutions
iv) adding the desired binder to the impregnated Zeolite; and
v) calcining the resultant mixture for 2-6 hrs in an oxygen containing atmosphere, preferably air, to produce the cracking catalyst particle.

22. The method according to claim 21, wherein the cracking catalyst and support are calcined at 300-1000° C., preferably at 350-800° C. and most preferably at 450-550° C. for 2-6 hrs in an oxygen containing atmosphere, preferably air.

25. A process for cracking hydrocarbons, comprising:
passing a mixture of hydrocarbons comprising 4 to 100 carbon atoms into a reaction chamber, wherein the reaction chamber comprises the cracking catalyst according to claim 1;
reacting the mixture of hydrocarbons over the cracking catalyst at a reaction temperature of 500-800° C., a weight hourly space velocity of 1-100 hr⁻¹, and a pressure of 0.01-0.2 MPa; and
converting, by wt %, at least 55% of the hydrocarbons in the mixture of hydrocarbons with a propylene selectivity of at least 30 wt %, and a propylene to ethylene mass ratio of at least 1.

24. (canceled)

25. (canceled)
